(19) United States
(12) Patent Application Publication
(10) Pub. No.: US 2010/0297027 A1
(43) Pub. Date: Nov. 25, 2010

(54) Overt authentication features for compositions and objects and methods of fabrication and verification thereof

(75) Inventors: Cedric Loiret-Bernal, Evanston, IL (US); Sylvain Cruchon-Dupeyrat, Chicago, IL (US); Bjoern Rosner, Chicago, IL (US)

Correspondence Address: FOLEY AND LARDNER LLP;SUITE 500, 3000 K STREET NW, WASHINGTON, DC, 20007, US

(73) Assignee: Nanolnk, Inc.

(21) Appl. No.: 11/305,189

(22) Filed: Dec. 19, 2005

Related U.S. Application Data
(60) Provisional application No. 60/637,064, filed on Dec. 20, 2004.

(51) Int. Cl. A61K 9/00; A61P 43/00

(52) U.S. Cl. 424/10.2; 424/10.1

(57) ABSTRACT

Overt anti-counterfeiting features on objects and compositions, especially pharmaceutical compositions. In a preferred embodiment, a counterfeiting-proof overt feature that presents a visually distinctive optical variable or invariable effect is manufactured on a pharmaceutical composition using a purely physical process. Further preferred are counterfeiting-proof, overt features that comprise at least one engineered array of micro- or nanostructures. Also provided are methods and apparatus for the manufacturing of said overt features on said objects and compositions, including pharmaceutical compositions, and for verifying the authenticity of said features. The inventive features do not reside in the method of making the stamp or the instrument used for stamping.

### RELATED APPLICATIONS

This application claims priority to provisional application serial no. 60/637,064 filed Dec. 20, 2004 to Cruchon-Dupeyrat et al. which is incorporated by reference in its entirety.

### BACKGROUND

The Food and Drug Administration (FDA) requires that “no drug product in solid oral dosage form” e.g., a tablet or capsule that contains a drug substance, “may be introduced . . . unless it is clearly marked or imprinted with a code imprint that, in conjunction with the product's size, shape, and color, permits the unique identification of the drug product and the manufacturer or distributor of the product.” See Code of Federal Regulations Title 21, Section 206.10. Possible code imprints include but are not limited to any single letter or number or any combination of letters and numbers, including, e.g. words, company name, and National Drug Code, or a mark, symbol, logo, or monogram, or a combination of letters, numbers, and marks or symbols, assigned by a drug firm to a specific drug product. Such code imprints are usually coarsely embossed, debossed, engraved, or printed with ink onto or into said drug product as part of or after the manufacturing process.

However, these conventional means of identification of pharmaceutical compositions are not counterfeiting-proof: the necessary tablet manufacturing equipment is readily commercially available; the fabrication methods are well-known to the art and openly published; and knowledge of the fabrication method and parameters may be easily derived from direct observation of the code imprint. Counterfeiters are known to readily produce fake or adulterated pharmaceutical compositions that mimic the genuine items, including any unit-level indicia, to a high degree of perfection, in order to deceive the end consumer or retailer. There is therefore a need to provide code imprints for pharmaceutical compositions, the appearance of which is not easily copied, to discourage criminal counterfeiting.

More generally, there is a need for counterfeiting-proof, overt features (“overt” meaning manifest, readily perceived by the senses and especially by the sight) on objects and compositions of commercial value, and especially pharmaceutical items. Said overt features may identify a brand, model or dose, as described above; they may comprise a bar code, a textual label, for example a serial number or a batch number, which identify a particular lot or unit. It may also be used as means of authentication of said objects and compositions or the information they bear.

Preferably, the counterfeiting-proof overt mark or code imprint should have a distinctive quality that is immediately obvious to the end consumer or retailer (possibly with the help of a simple instrument, device or kit) but not easily copied. It is especially advantageous to prepare overt marks that have a distinctive visual appearance, for example a specific hue or opalescent appearance.

Furthermore, duplication of said distinctive quality (with possession of an original item or not) should be difficult or costly to implement, and ideally only possible with exact knowledge/ownership of the original fabrication methods, apparatus and materials. The fabrication method and fabrication parameters of said overt marks should not be easily derived from direct observation of the mark. The design and operation of the necessary patterning and manufacturing equipment should preferably require specialized skills and technologies not ordinarily available to counterfeiters. The cost of manufacturing, however, should be kept low, e.g., in the order of cents per unit.

Therefore, a need exists to provide anti-counterfeiting overt marks that present the aforementioned qualities, as well as methods of use and fabrication thereof and apparatus therefor. In particular, a need exists to provide counterfeiting-proof overt marks for pharmaceutical compositions that derive their distinctive visual appearance from micrometer-scale to nanometer-scale patterns, and especially from engineered collections or arrays thereof. “Engineered collections or arrays” include but are not limited to regular or semi-regular arrangements of patterns. Examples include gratings of lines, rectangular or hexagonal arrays of dots or squares, but also pseudo-random distributions of patterns. Array of pixels, each pixel itself being an array of micro- or nanoscale patterns, are also included. Micrometer-scale or smaller patterns are of interest, because they are typically neither easily reproduced with conventional pharmaceutical manufacturing equipment, nor individually observable with the naked eye; yet collectively, a properly designed array thereof may have a strong optical effect.

Micrometer-scale pattern arrays having a distinctive visual appearance for anti-counterfeiting are known to the art. Examples include holographic logos on credit cards, passports, and banknotes. The document entitled “Pharmaceutical counterfeiting, tampering and diversion: The threat to world health and the quest for solutions” from American Bank Note Holographics (http://www.abnh.com/security/Pharm_WhitePaper_WEB SITE.df) describes solutions for the pharmaceutical industry, including HoloSeal™ holographic labels, HoloSleeve™ holographic anti-tampering heat-shrink sleeves that may be applied to the necks of bottles and containers, and HoloCap™ induction-sealed cap seals. Companies like Applied Optical Technologies (Washington, Tyne & Wear, UK) and OVD Kinegram (Zug, Switzerland) also manufacture similar anti-counterfeit labels, tamper-evident closures, and blister foils that display anti-counterfeiting imagery.

However, a need exists to experimentally apply theoretical knowledge of the (optical) properties of micrometer- and nanoscale patterns to the unit-level marking of pharmaceutical compositions, such as tablets and hard gelatin capsules, or of edible labels applied to pharmaceutical compositions.

In addition, there is also a need for a method of fabrication of identification marks on pharmaceutical compositions, which is intrinsically safe and compatible with pharmaceutical regulations.

Prior methods in the art have marked pharmaceutical compositions with e.g. pearlescent inks, molecular or isotopic taggants, color-shifting dyes, etc . . . For example, November AG (Erlangen, Germany) and Applied DNA sciences (Los Angeles, Calif.) sell synthetic and natural DNA markers, respectively. In many cases, however, such prior methods are inadequate for the identification of edible items. Because these methods often require the addition, deposition or printing of (an) exotic or unapproved chemical compound(s), they may carry significant health risk or may delay the approval of or trigger a reexamination of the targeted drug product by regulatory authorities.

Therefore, a need exists to provide a method of overt mark manufacturing that requires neither a significant chemical modification of the pharmaceutical composition, nor any addition to the pharmaceutical composition or its coating. Preferably, a need exists to provide overt marks by a physical process, such as the (thermo)mechanical modification of the topography of the coating of the drug product e.g. by shallow embossing, debossing or engraving.

### SUMMARY

Exemplary embodiments of the present invention are summarized in this non-limiting section. The invention, in its various embodiments, enables one to prevent counterfeiting, tampering and other fraud. In particular, overt counterfeiting-proof regions on objects and compositions are provided, especially pharmaceutical compositions, such as tablets and capsules.

The invention generally relates to counterfeiting-proof, overt identification or authentication marks on objects and compositions, especially pharmaceutical compositions. It further relates to methods and apparatus for the manufacturing of said overt features and for verifying the authenticity of said features.

A pharmaceutical composition comprising:

One embodiment provides a pharmaceutical composition comprising at least one identification region, said region comprising at least two features, each feature having at least one lateral dimension of about 100 microns or less, the features collectively creating at least one optical effect that may be readily observed with the naked eye.

Another embodiment provides a pharmaceutical composition comprising: a pharmaceutical composition comprising at least one identification region, said region comprising at least two features, each feature having at least one lateral dimension of about 100 microns or less, the features collectively creating at least one optical effect that may be readily observed with the naked eye or with the help of a device or kit. The identification region of the stamp can be contoured.

For purposes of this application, the inventive features do not reside in the method of making the stamp or the instrument used for stamping.

### BRIEF DESCRIPTION OF THE DRAWINGS

FIG. 1 is a schematic diagram of a microstructured stamp containing a logo. The size of all features together is approximately 1 mm by 3 mm.

FIG. 2 is a scanning electron micrograph of an overt mark embossed in a commercial pharmaceutical tablet using the stamp of FIG. 1 but broken into a smaller 1 mm by 1 mm piece.

FIG. 3 is an optical microscopy image of the same overt mark.

FIG. 4 illustrates the hot-embossing of a grating comprising an array of micrometer-scale squares into a commercial pharmaceutical preparation. A is an atomic force microscope image of the stamp, while B shows the surface of the tablet after embossing.

FIG. 5 illustrates the diffraction of a red laser onto a commercial tablet. [A] shows the reflection off an unmodified part of the tablet, while [B] shows the reflection off a microstructured part of the surface of the tablet.

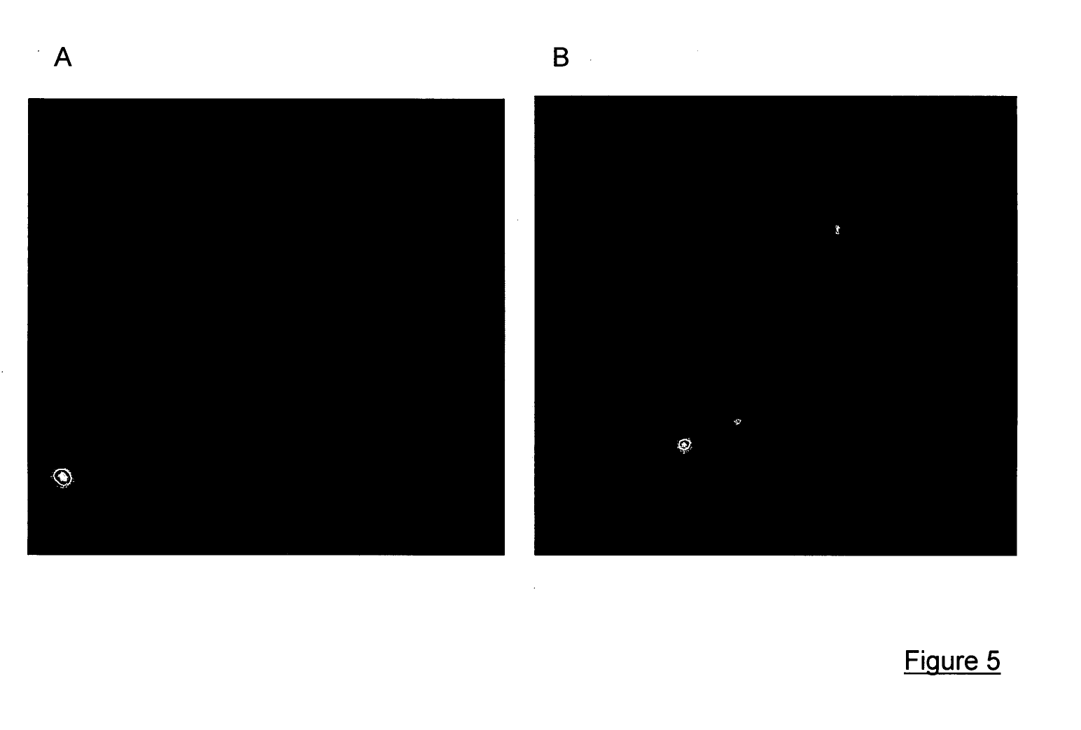

### DETAILED DESCRIPTION OF THE PREFERRED EMBODIMENTS

The references cited in this or any other section can be referred to in the practice of the present invention and are hereby incorporated by reference in their entirety. No admission is made that any of them is prior art.

The invention provides counterfeiting-proof, overt features on objects and compositions of commercial value, and especially pharmaceutical items.

In one embodiment, the invention provides a pharmaceutical composition comprising:

a pharmaceutical composition having a surface, wherein the surface comprises at least one overt identification region, the overt identification region having at least two identification features, wherein the features each have lateral dimensions of about 100 microns or less and form an overt identification region.

In one embodiment, the invention provides a pharmaceutical composition having a surface, wherein the surface comprises at least one identification region, the region having at least two features, the features having a lateral dimension below about 100 microns (and especially 15 micron or less, and especially 1.5 micron or less and especially 0.6 micron or less), the features collectively creating at least one distinctive visual appearance that may be readily observed with the naked eye or with the help of a simple device or kit. It may be a distinctive hue; it may comprise a variable optical effect i.e. a hologram or an opalescent region. Alternatively, the identification region may induce a moire effect when observed with the proper device or it may polarize light. In a preferred embodiment, the identification region comprises an engineered collection or array of micrometer to nanometer-scale patterns, for example line segments or dots.

The invention also provides overt identification marks directly imprinted on medical devices, implants, catheters, stents, syringes, injectable drug vials, pharmaceutical tablet container, excluding heat-shrink seals, bottle cap seals and labels.

Overt marks can be prepared including marks 1 mm×1 mm or less, or 1 mm square or less.

The invention further provides methods and apparatus for the manufacturing of said overt features on said objects and compositions, and especially pharmaceutical compositions.

For example, the invention provides a method for the direct imprinting of a pharmaceutical composition with at least one counterfeiting-proof overt mark without use of ink, which comprises the step of contacting the pharmaceutical composition with a microstructured stamp (to emboss/deboss, imprint or engrave said composition). In a preferred embodiment, the pharmaceutical composition is hot-embossed with a hard stamp to a shallow depth, so that only the coating of the pharmaceutical composition is affected. In another example, the pharmaceutical composition is printed with ink. The method for fabricating a stamp adapted to the imprinting of pharmaceutical compositions, said stamp comprising contoured, three-dimensional microrelief structures. In another embodiment, the invention provides a pharmaceutical composition comprising at least one counterfeiting-proof overt mark and at least one covert mark. The covert mark may be used for the forensic authentication of or information storage on the pharmaceutical composition. Finally, the invention provides methods, apparatus and kits for verifying the authenticity of identification features on a pharmaceutical composition, if it may not be verified with the naked eye.

### EMBODIMENT 1

### Pharmaceutical Composition Having Overt Counterfeiting-Proof Mark

In a first embodiment, the invention provides a pharmaceutical composition comprising:

a pharmaceutical composition having a surface, wherein the surface comprises at least one overt identification region, the overt identification region having at least two identification features, wherein the features each have lateral dimensions of about 100 microns or less and form an overt identification region. The lateral dimension can be about 10 microns or less, about one micron or less, about 500 nm or less, or about 100 nm or less.

In addition, the invention also provides a pharmaceutical composition having a surface, wherein the surface comprises at least one identification region, the region having at least two features, each feature having a lateral dimension below about 100 microns, the features collectively creating at least one distinctive effect that may be readily observed but not easily copied.

In a preferred embodiment, the distinctive effect is optical in nature and is readily observed with the naked eye or with the help of a simple device or kit, such as an optical filter or a portable microscope.

While it is advantageous to prepare overt marks that have a distinctive visual appearance, the overt mark aspect may appeal to other senses. For example, the overt mark may provide a tactile sensation: the surface of the pharmaceutical composition may be textured. Alternatively, effects may be combined. The overt mark may comprise fragile structures that are easily damaged by the touch of one's fingers and which visual appearance would change irreversibly thereafter.

The distinctive effect of the overt mark should not be easily reproduced by conventional means and methods of manufacture. Duplication of the effect (even when in possession of an original item) should be difficult or costly to implement, and ideally only possible with exact knowledge or ownership of the original fabrication methods, apparatus and materials. The structure or morphology of the overt mark should not be easily deduced from said visual appearance. The fabrication method and fabrication parameters of said overt marks should not be easily derived from direct observation of the mark. The design and operation of the necessary patterning and manufacturing equipment should preferably require specialized skills and technologies not ordinarily available to counterfeiters.

The distinctive effect of the said identification region may be permanent or it may vary with time or in response to external stimuli. For example, the overt mark may be so designed that its appearance is irreversibly modified upon exposure to excessive heat, humidity or vibrations, thus providing protection against mishandling or abuse.

In a preferred embodiment, at least one of the lateral dimensions of said features in said identification region is about 15 micron or less, and especially about 1.5 micron or less, and especially about 0.6 micron or less. The vertical dimension may be about 10 microns or less, more particularly, about 5 microns or less, more particularly, about 1 micron or less, and especially it may be about 0.3 micron or less or about 0.1 micron or less. The pitch (distance) between the features in said identification region may be about 15 microns or less; and especially it may be about 1.5 microns; further, it may be about 0.6 microns or less. The lateral dimension, vertical dimension and pitch may be simultaneously about 1.5 micrometer or less.

“Lateral dimensions” can be measured substantially parallel to the surface of the pharmaceutical composition in the area of the overt code. The “vertical dimension” can be measured in a direction substantially normal to said surface and thus usually corresponds to a depth or height. If the surface of the pharmaceutical composition is curved, measurements are taken following the curvature.

In a further preferred embodiment, the invention provides counterfeiting-proof overt marks for pharmaceutical compositions that derive their distinctive visual appearance from engineered collections or arrays of micrometer-scale to nanometer-scale patterns.

“Engineered collections or arrays” include but are not limited to regular or semi-regular arrangements of patterns. Examples include gratings of lines, rectangular or hexagonal arrays of dots or squares, but also pseudo-random distributions of patterns (“pseudo-random” meaning that, while the patterns may seem placed at random, their spatial frequency distribution is known and engineered to obtain a desired optical effect). Said overt mark may comprise features at multiple length scales, for example an array of pixels, which may be a small square region (for example 30×30 um²), each pixel itself comprising an array of micro- or nanoscale patterns. Possible optical effects and manners to produce them are briefly reviewed in a later section.

The identification region may comprise one or more features, which protect the information-bearing part from erasure or damage. For example, a raised ring or frame surrounding the identification features may avoid mechanical abrasion of the identification.

The pharmaceutical composition on which to form the overt mark is not particularly limited and can be useful for human or animal treatment. A wide variety of pharmaceutical compositions are known in the art including pharmaceutical drugs in various shapes and sizes such as pills, tablets, capsules, and the like. For example, tablets and caplets can be used. The overt mark may be added at various steps of the manufacturing process, for example after compressing or coating. Two-piece (hard gelatin) capsules can be used, before or after filling with powder, gel, or liquid. Pill surface structures can include sugar shell, soft-shell, dipped or enrobed, enteric, or aqueous coated tablets, waxed tablets, and dry coatings. Smooth, optically reflective coatings are preferred.

The pharmaceutical compositions can contain active ingredients and inactive ingredients, and in different embodiments, these can be distributed differently. The pharmaceutical composition can have an exterior region or surface which can be processed to include one or more identification regions by, for example, imprinting or embossing, whether hot or cold embossing. Hence, the pharmaceutical composition can be processible and susceptible to, for example, heat and pressure effects which allow for imprinting or embossing. The pharmaceutical composition generally can comprise an active pharmaceutical ingredient (API) but the invention is not particularly limited to how the API is distributed throughout the pharmaceutical composition. For example, the API could be in the interior or could be subjected to a coating process. A composition could be provided with the overt feature and then combined with the API. Hence, for example, the surface of the pharmaceutical composition may have little if any of the API but yet it is still part of the pharmaceutical composition. The API can be a solid, liquid, or gel API as long as the ultimate pharmaceutical composition can be processed to include the identification features described herein.

The surface of the pharmaceutical composition can be an exterior surface which represents an interface with air. In addition, however, the surface of the pharmaceutical composition could be an interior surface. For example, an interior surface can be prepared by generating a desired surface having desired surface features and then combining that surface with another composition so that the desired features are no longer directly exposed to the air but can be detected, even though they are now interior surfaces. For example, an identification region can be generated and then overcoated with a protective film, coating, or layer, which include but are not limited to thin conformal films. The surface of the pharmaceutical composition can be generally flat and smooth, although at the scale of the identification features described herein the surface can be generally rougher. Or the surface can be non-flat or curved, including spherical, oval, or bi-convex. An interior surface can be desirable to avoid scratching or rubbing of the identification region.

The design of the overt mark may be kept throughout the lifecycle of the particular pharmaceutical product; alternatively, all or parts of it may be changed from time to time, in order to deny counterfeiters enough time for duplication or to convey a time-sensitive message (expiration date of the pharmaceutical unit, for example). Optionally, the overt mark may be customized for a given market segment, in order to prevent diversion of the drug product (illegal or undesired reimportation, for example). For example, the overt mark may comprise the name of the intended destination country; or it may be a word label such as “sample”, “clinical trial” or the name of a charitable or humanitarian organization to which the pharmaceutical composition has been given.

### Optical Effects

The overt counterfeiting-proof mark of embodiment 1 may have one among several distinctive optical effects, depending on the arrangement of said features within said identification region. For example, if said features form a regularly spaced array, the optical signatures of said overt mark may depend on the width, pitch, depth of said patterns.

For example, said overt marks may present a distinctive hue or light absorption characteristics (e.g. a gray, black color; or conversely a highly reflective appearance); they may also present a variable, angle-dependent optical effect (e.g. rainbow or opalescent effect) or even a holographic image produced by one or more diffraction gratings or diffractive patterns.

The overt mark may present a moire effect when observed through the proper filter or may polarize light in a way that is easily detected with an appropriate analyzer. These effects may be present on all or parts of the overt mark (or surrounding areas) and may be combined with each other.

The following presents background information that may be used to practice the invention, esp. with respect with the engineering of said micrometer-scale patterns to produce the desired effect in said overt mark. Said background information is hereby incorporated by reference in its entirety:
1) “Microtechnology for anticounterfeiting” R. Lee, Microelectronic Engineering 53, 513-516 (2000), which describes the use of electron-beam lithography to produce diffractive or micrographic optical microstructures on e.g. security papers and banknotes;
2) “Microrelief structures for anti-counterfeiting applications”, P. Leech, H. Zeidler, Microelectronic Engineering 65, 439-446 (2003), which provides examples of 3D relief structures intended for the direct printing of specialized inks onto documents;
3) “Fabrication of hologram coins using electron-beam lithography” P. Leech et al. Microelectronic Engineering 71, 171-176 (2004), which describes currency impressed with a relief microstructure forming an optically variable device.
4) “Colourtone lithography” R. Lee, Microelectronic Engineering 61-62, 105-111 (2002), which presents advanced pixellated microstructure arrays, including micromirror arrays.

A review of the theory of diffraction gratings may be found in standard optics texts as well as in the “diffraction grating handbook” by Christopher Palmer (Erwin Loewen, Ed.) published by the Richardson Grating Laboratory, now part of Newport Corp. (Mountain View, Calif.). A short introduction on the subject may also be found in the Jobin-Yvon optics tutorial (http://www.jobinyvon.com).

Computer software may also be used to calculate the parameters of a diffraction grating and automatically designing a grating with the desired characteristics. The GSOLVER© software from Grating Solver Development Company, Allen, Tex. is one example of such software (http://www.gsolver.com).

Furthermore, European Patent EP 0 449 893 to Lee et al., entitled “diffraction grating”, and European Patent EP 0490 923 to Lee disclose diffraction grating devices employed in security devices and especially arrays having pixellated diffraction grating portions, in which each pixel is an individual diffraction grating of curvilinear lines. Theory is provided to afford structurally stable holographic images. U.S. Pat. No. 5,729,365 to Sweatt (Sandia Corp., 1996) describes a computer-generated microlithographic tag or microtag, which are covert phase grating identification marks with extremely small features written by extreme UV lithography.

With regards to optical black or grey structures, PCT patent application WO 03/098188 to Steenblik et al. (Nanovention), which is entitled “Microstructured taggant particles, applications and methods of making the same” and especially page 6, as well as U.S. application Ser. No. 10/351,285 can be employed. Briefly, an array of high-aspect-ratio microstructures may trap light by multiple glossy reflections between adjacent elements, until all light has been absorbed.

With reference to polarization effects, the reader is referred to the NanoOpto, Inc. white paper entitled “Nano-optics: changing the rules for optical system design” by H. Kostal, and in particular its mention of subwavelength grating structures—grating with one or more dimensions one or more order of magnitude smaller than the wavelength of light, typically in the order of tens to hundreds of nanometers. Appropriate selection of the dimensions of the grating affords a polarizer, waveplate or a polarization-dependent filter. It is expected that the difference in the polarization behavior of nanostructured regions and non-nanostructured regions, which behave according to Fresnel's reflectance law may be identified with a polarizing filter acting as an analyzer.

With regards to color generation due to diffractive, interference or scattering effects, one may refer to “Nano-optics in the biological world: Beetles, butterflies, birds, and moths” Srinivasarao, Chemical Reviews 99 (7): 1935-1961 (1999) and “Angle-dependent optical effects deriving from submicron structures of films and pigments” Chem. Rev. 99, 1963 (1999). For example, the wings of Blue Morpho butterflies are known for their iridescent structural colors that may be viewed over a large angle range due to interference or diffractive effects caused by their microstructures. Artificial diffraction gratings can imitate that effect when composed, for example, of many small hexagonal gratings, each inscribed with tiny grooves aligned in one of six directions. An example grating with a spacing of 440 nm and a depth of 125 nm was demonstrated by electron beam lithography and etching, see e.g., “Butterfly blues”, The Industrial Physicist, April/May 2004, page 13, Optics Lett. 2003, 28, 2342.

The overt mark may also be a watermark i.e. a marking at the surface of the pharmaceutical composition resulting from differences in thickness, which is usually produced by pressure of a mold or on a processing roll.

Regarding the Moire effect, literature to practice the invention includes the U.S. patent application 2002/0054680 to Huang et al. (TrustCopy Inc.). The overt mark may comprise, for example, an array of generally regularly spaced dots. Information may be encoded as small variations in the position of groups of dots and is read using a decoder key comprising a matching array of dots.

Finally, Nanoventions' U.S. patent application 2003/0179364 to Steenblik et al. entitled “Microoptics for article identification” discloses “distinctive optical effects obtained from micro-optic systems incorporating micro lenses, non-imaging collectors, prisms, wave guides, mirrors, gratings, structural interference filters, and photonic crystal microstructures”.

The present invention improves on the aforementioned designs and structure by incorporating them onto pharmaceutical compositions to provide a distinctive optical effect.

### EMBODIMENT 2

### Method to Detect Counterfeited Pharmaceutical Compositions

Methods to identify or track pills are known to the art. For example, U.S. Pat. No. 6,543,692 to Nelhaus et al. describes a scheme for the identification of solid-form drugs, which combines a common visual symbol that is easily recognized and a machine-readable bar code. U.S. Pat. No. 5,992,742 to Sullivan et al. discloses a machine-readable code on a label of a pill or imprinted directly thereon. U.S. Pat. No. 6,799,725 to Hess et al. discloses a (micro-barcoded) pill identification system.

While said identification and tracking systems may incidentally detect simple-minded counterfeiting, to the best of our knowledge, little is known about a reliable method to systematically detect counterfeiting at the unit level.

Therefore, in a second embodiment, the invention provides a method for detecting counterfeited pharmaceutical compositions in a market, said method comprising the steps of:
(i) providing a pharmaceutical composition with a unit-level overt authenticating mark, said mark having at least one distinctive quality observable with the naked eye or with a simple kit or apparatus,
(ii) optionally providing said kit or simple apparatus to read or decode said overt mark,
(iii) distributing said pharmaceutical composition and optionally the kit or apparatus on a market,
(iv) educating the end user or retailer in said market about (a) the presence and role of the overt mark(s); and (b) methods for reading the mark, including the optional use of said reading kit or apparatus,
   The end user or e.g. pharmacist, once made aware of the existence of the overt mark(s) by the proper documentation (e.g. advertisements or FDA-required prescription pamphlet), will recognize and expect the presence of the overt mark. The absence thereof will alert him or her about the possibility of a fake or error.
(v) optionally providing means for end user or retailer to give feedback regarding units without the overt mark or with an obviously forged mark.
   Because the end consumer is involved, the method above creates a fine-grained, early-warning counterfeiting detection network. In addition, the presence of an overt mark on pharmaceutical units may positively associate a brand or dose with the notion of safety and security.

### EMBODIMENT 3

### Methods of Fabrication of Overt, Counterfeiting-Proof Marks On Pharmaceutical Compositions

In a third embodiment, the invention provides a method for the direct imprinting of a pharmaceutical composition with at least one counterfeiting-proof overt mark without use of ink, the method comprising the steps of:
providing a stamp, which has a surface adapted to form at least one region comprising at least one engineered collection of features, the lateral size of said features being inferior to about 100 microns;
providing a pharmaceutical composition having a surface;
and contacting the stamp and the pharmaceutical composition.
   where the engineering collection of patterns, once transferred, may create at least one distinctive effect, especially an optical effect that is readily observed with the naked eye or with the help of a simple device or kit.

This method neither adds a chemical substance (e.g. an ink or a dye) to the pharmaceutical unit, nor significantly modifies its chemical composition.

### Hot Embossing

In a preferred embodiment, the invention provides a method for the fabrication of overt marks in pharmaceutical compositions, the mark having at least one region, the region having at least two features of a lateral dimension inferior to about 100 microns, the method comprising the steps of:
providing a hard stamp, which has a surface adapted to form at least one region comprising at least two features, the lateral size of said patterns being inferior to about 100 microns;
optionally heating the stamp to a desired temperature,
providing a pharmaceutical composition having a surface;
and contacting the stamp and the pharmaceutical composition,
   Preferably, the pharmaceutical composition is only shallowly embossed, so that only its external coating is affected.

In a further preferred embodiment, the invention provides the method described above, further comprising the use of a stamp which surface is contoured, three-dimensional in nature. This is especially useful for the fabrication of blazed gratings, including for example gratings with triangular grooves.

One takes note of the following related art, which may be used to practice the invention, especially with respect to the preparation of a master, the preparation of the necessary stamp(s) from said master and the stamping tools:
1) U.S. Pat. No. 6,006,415 to Schaefer et al. entitled “Transfer for transferring holograms onto metal surfaces”,
2) U.S. Pat. No. 6,375,870 to Visovsky et al., which is entitled “Replicating a Nanoscale Pattern”. It provides an apparatus comprising a cylindrical roller. The cylindrical roller can be adapted to carry a micro- or nanoscale pattern on its outer surface and transfer said pattern to a substrate.
3) U.S. Pat. No. 6,651,338 to Helm entitled “method for the production of a cylindrical embossing sheet”.
4) U.S. Pat. No. 6,694,873 to Labelle et al. entitled “Microembosser for faster production of holographic labels”

### Printing

In an alternative embodiment, the invention provides ultra-high resolution printing of micrometer- to nanometer-scale features on said pharmaceutical composition, said features forming an engineered array. Specifically, the invention provides a method for printing on a pharmaceutical composition with use of ink, the method comprising the steps of:
providing a stamp coated with ink, which has a surface adapted to transfer ink to form at least one region comprising at least one engineered collection of patterns, the lateral size of said patterns being inferior to about 15 microns;
providing a pharmaceutical composition having a surface;
and contacting the stamp and the pharmaceutical composition,
   so that the printed region provides at least one distinctive optical effect that may be readily observed with the naked eye or with the help of a simple device or kit.

Inks can comprise nanoparticle solutions.

The following literature may be used to practice the invention and is hereby incorporated by reference in its entirety:
1) U.S. Pat. No. 5,006,362 to Hilborn, which is entitled “Branding pharmaceutical dosage forms, food and confectionery products with aqueous ingestible inks” provides a method of marking tablets, capsules . . . with inks. In particular, it describes suitable recipes for water-based, ingestible inks.
2) U.S. Pat. No. 5,512,131 to Kumar et al. entitled “Formation of microstamped patterns on surfaces and derivative articles”, which discloses microcontact printing.

### EMBODIMENT 4

### Stamp

The pharmaceutical compositions, as well as other objects and compositions, can be made with one or more stamps which provide the surface with the identification region. The invention provides a stamp for use in making a pharmaceutical composition, or other objects and compositions, the stamp comprising a surface having at least one identification region, and the region having at least two features. In general, the stamps can be master stamps and can be used repeatedly, or can be used to produce other stamps.

Because the stamp can have a shape which is transferred to the pharmaceutical composition, or other objects and compositions, the dimensions described herein for the identification features and identification regions can also be used to describe the stamp. For example, a stamp which has an identification feature having a 100 nm height can result in a pharmaceutical composition, or other composition or object, having an identification feature with 100 nm height. The stamp's 100 nm positive protrusion can produce a 100 nm recess.

The material of the stamp is not particularly limited. In general, the stamp can have a surface which is made of a harder or stiffer material than the material of the surface to be stamped. Materials that can provide high aspect ratio structures can be used. For example, materials can be used which can be subjected to etching processes which result in high aspect ratios such as, for example, reactive ion etching. Stamp material can be, for example, silicon, silicon oxide, silicon nitride, quartz, and nickel, and other substrates popular for semiconductor processing. In a preferred embodiment, titanium is used.

Stamps can be also treated to increase the durability of the stamp. For example, stamp surfaces can be coated with diamond like coatings (DLC), or nickel films to increase hardness and decrease wear. Stamps fabricated from silicon substrates can be oxidized to increase the hardness of the stamp surface.

A master stamp can be used to produce daughter stamps which are substantially identical but inverted copies of the master.

### EMBODIMENT 5

### Method of Fabrication of the Stamp

Methods are known in the art to fabricate the stamp of embodiment 4 and used in embodiment 3. For example, photolithography, optical lithography, electron-beam direct-write lithography, X-ray lithography may be used to generate micrometer-scale patterns (and even nanoscale patterns, in the case of e-beam and deep UV lithography) in a resist layer on a substrate. The pattern may be then transferred via etching methods, lift-off methods, electroless deposition, electroplating, etc... into a metallic or ceramic substrate suitable for embossing a pharmaceutical composition.

In a possible embodiment, micro- or nanolithography can be used to selectively remove or modify areas in a resist film coating a substrate. The patterned substrate can be subjected to etching to generate negative relief features, such as trenches, in the substrate. The resulting negative relief stamp can be used directly or can be replicated to generate secondary stamps that have positive features.

In another embodiment, the invention provides a method for fabricating (e.g. blazed) embossing stamps having a contoured, 3D surface. The method may comprise (a) the use of anisotropic etching, for example of silicon <100> in e.g. KOH, to create pits and trenches having sloped sidewalls. Another method for fabricating a stamp adapted to the imprinting of pharmaceutical compositions, said stamp comprising contoured, three-dimensional microrelief structures, involve the use of grey-scale optical lithography. Grey-scale lithography is available commercially, for example from Canyon Materials, Inc. (San Diego, Calif.). Yet another method for fabricating a master stamp involves the modification of commercial grating, which are available from e.g. Edmund Optics, Horiba Jobin-Yvon and Thorslabs. In a possible process, a commercial grating is coated with a thick resist; the resist is selectively exposed to UV radiation in e.g. a mask aligner; and unwanted portions of the grating are etched away to form the desired stamp. Methods for the fabrication of the starting holographic grating are disclosed, for example, in U.S. Pat. Nos. 5,182,659 and 5,270,842 to Clay et al., which are hereby incorporated by reference in their entirety.

3D laser micromachining of a polymer substrate followed by formation of a daughter stamp via electroplating is also a possible process, although at low resolution. Such service is available from e.g. Potomac Laser Microtools corp. Nanoimprint lithography may also be used for triangular profile imprint mold fabrication, see e.g., Yu, Chou Nanoletters 4(2), 341, 2004.

### EMBODIMENT 6

### Other Objects or Compositions Having an Overt Identification Mark

Those skilled in the art will acknowledge the existence of multiple functionally equivalent variants of the present embodiments which shall not be considered distinct from the present invention.

In another embodiment, the invention also provides an object comprising:

an object having a surface, wherein the surface comprises at least one overt identification region, the overt identification region having at least two identification features, wherein the features each have lateral dimensions of about 100 microns or less and form an overt identification region. The lateral dimension can be about 10 microns or less, about one micron or less, about 500 nm or less, or about 100 nm or less.

For example, discrete consumable items, especially food and confectionery items, such as candy in discrete or extruded form, mints, balls, gums, strips and the like, may be marked with overt regions having a distinctive visual appearance, such as an iridescent pattern or a holographic image. Overt marks on food items may be provided as means of identification but also for aesthetic appeal and branding purposes.

In a second example, overt features on medical devices and supplies, especially these with a thin external polymeric coating, are covered by the present invention. The invention provides overt identification marks directly imprinted on medical devices, implants, catheters, stents, syringes, injectable drug vials, pharmaceutical tablet container, with the exclusion of bottle seals, labels, and metal containers. Direct embossing of said items or of their external coatings may be preferred to the affixing of an anti-tampering label or heat-shrink sleeve, because the later can usually be removed without leaving a trace.

### SERIES OF PREFERRED EMBODIMENTS

An additional 31 preferred embodiments are specified.

1. A pharmaceutical composition comprising:

at least one identification region, the identification region comprising at least two features, each of the two features having at least one lateral dimension less than about 100 microns, the two features collectively creating at least one distinctive optical effect.

2. A pharmaceutical composition according to 1, wherein the optical effect is observable with the naked eye.

3. A pharmaceutical composition according to 1, wherein the optical effect is observable with the aid of a device.

4. A pharmaceutical composition according to 1, wherein the optical effect is observable with the aid of an optical filter, a microscope, an angled light source or a light analyzer.

5. The pharmaceutical composition according to 1, wherein each of the two features has at least one lateral dimension less than about 15 microns.

6. The pharmaceutical composition according to 1, wherein each of the two features has at least one lateral dimension of about 1.5 microns or less.

7. The pharmaceutical composition according to 1, wherein each of the two features has at least one lateral dimension less than to about 0.6 microns.

8. The pharmaceutical composition according to 1, where the distinctive optical effect comprises an invariable optical effect.

9. The pharmaceutical composition according to 1, wherein the distinctive optical effect comprises a distinctive hue that does not solely result from the presence of a dye or pigment.

10. The pharmaceutical composition according to 1, wherein the distinctive optical effect comprises absorbing light in a way that does not solely result from the presence of a dye or pigment.

11. The pharmaceutical composition according to 1, wherein the distinctive optical effect is a variable optical effect.

12. The pharmaceutical composition according to 1, wherein at least one identification region comprises a hologram.

13. The pharmaceutical composition according to 1, wherein at least one identification region comprises an opalescent overt identification region.

14. The pharmaceutical composition according to 1, wherein the distinctive optical effect comprises polarizing reflected light.

15. An anti-counterfeiting overt mark within or at the surface of a pharmaceutical composition, the mark comprising at least one engineered collection of micrometer-scale to nanometer-scale patterns, the lateral size of the patterns being less than about 100 microns.

16. The mark according to 15, wherein the engineered collection is a periodic array.

17. The mark according to 16, wherein the pitch between patterns is less than about 1.5 microns.

18. The mark according to 16, wherein the periodic array comprises a grating of substantially parallel lines.

19. The mark according to 16, wherein the engineered collection of patters comprises at least one array of dots or squares.

20. The mark according to 15, wherein the engineered collection of patterns comprises a pseudo-random distribution of micro- or nanometer-scale patterns.

21. An article comprising at least one overt identification region, wherein the overt identification region comprises at least two features, each of the two features having at least one lateral dimension less than about 30 microns, the two features collectively creating a distinctive optical effect, and wherein the article is selected from the group consisting of medical devices, implants, catheters, stents, syringes, injectable drug vials, pharmaceutical tablet containers, excluding bottle seals and labels.

22. An article according to 21, wherein the distinctive optical effect is observable with the naked eye.

23. An article according to 21, wherein the distinctive optical effect is observable with the aid of a device.

24. An article according to 21, wherein the distinctive optical effect is observable with the aid of an optical filter, a microscope, an angled light source or a light analyzer.

25. A method for directly imprinting a pharmaceutical composition with at least one overt, anti-counterfeiting mark without the use of ink, the method comprising:

(a) providing a stamp, which has a surface adapted to form at least one region comprising at least one engineered collection of patterns, the lateral size of the patterns being less than about 100 microns;

(b) providing a pharmaceutical composition having a surface; and

(c) contacting the stamp and the surface of the pharmaceutical composition.

26. A method according to 25, wherein the stamp comprises a hard, heated stamp.

27. The method according to 25, wherein the surface comprises a contoured, three-dimensional surface.

28. A method for printing at least one overt, anti-counterfeiting mark onto a pharmaceutical composition using an ink, the method comprising:

(a) providing a stamp coated with ink, wherein the stamp has a surface adapted to transfer ink to form at least one region comprising at least one engineered collection of patterns, the lateral size of the patterns being less than about 15 microns;

(b) providing a pharmaceutical composition having a surface; and

(c) contacting the stamp and the surface of the pharmaceutical composition, so that the printed region provides at least one distinctive optical effect.

29. An article according to 28, wherein the distinctive optical effect is observable with the naked eye.

30. An article according to 30, wherein the distinctive optical effect is observable with the aid of a device.

31. An article according to 30, wherein the distinctive optical effect is observable with the aid of an optical filter, a microscope, an angled light source or a light analyzer.

### FURTHER LITERATURE TO PRACTICE THE INVENTION

One preferred lithography method is use of a sharp tip to transfer a patterning compound to a substrate to form patterns which can be used in fabrication of stamps. The following patents and co-pending applications are hereby incorporated by reference in their entirety:
1. U.S. Provisional Application 60/115,133 filed Jan. 7, 1999 (“Dip Pen Nanolithography”) to Mirkin et al.
2. U.S. Provisional Application 60/157,633 filed Oct. 4, 1999 to Mirkin et al. (“Methods Utilizing Scanning Probe Microscope Tips and Products Therefor or Produced Thereby”)
3. U.S. Regular Patent Application 09/477,997 filed Jan. 5, 2000 (“Methods Utilizing Scanning Probe Microscope Tips and Products Therefor or Produced Thereby”) to Mirkin et al.
4. U.S. Provisional Application 60/207,713 filed May 26, 2000 (“Methods Utilizing Scanning Probe Microscope Tips and Products Therefor or Produced Thereby”) to Mirkin et al.
5. U.S. Provisional Application 60/207,711 filed May 26, 2000 (“Methods Utilizing Scanning Probe Microscope Tips and Products Therefor or Produced Thereby”) to Mirkin et al.
6. U.S. regular application 09/866,533 filed May 24, 2001 (“Methods Utilizing Scanning Probe Microscope Tips and Products Therefor or Produced Thereby”) to Mirkin et al.
7. U.S. Patent Publication 2002/0063212 A1, published May 30, 2002 (“Methods Utilizing Scanning Probe Microscope Tips and Products Therefor or Produced Thereby”) to Mirkin et al.
8. U.S. Patent Publication 2002/0122873 A1 published Sep. 5, 2002 (“Nanolithography Methods and Products Produced Therefor and Produced Thereby”).
9. PCT Publication WO 00/41213 A1 published Jul. 13, 2000 based on PCT application no. PCT/US00/00319 filed Jan. 7, 2000 (“Methods Utilizing Scanning Probe Microscope Tips and Products Therefor or Produced Thereby”).
10. PCT Publication WO 01/91855 A1 published Dec. 6, 2001 based on PCT application no. PCT/US01/17067 filed May 25, 2001 (“Methods Utilizing Scanning Probe Microscope Tips and Products Therefor or Produced Thereby”).
11. U.S. Regular patent application, Ser. No. 10/307,515 filed Dec. 2, 2002 to Mirkin et al. (“Direct-Write Nanolithographic Deposition of Nucleic Acids from Nanoscopic Tips”).
12. U.S. Regular patent application, Ser. No. 10/320,721 filed Dec. 17, 2002 (“Patterning of Solid State Features by Direct-Write Nanolithographic Printing”) to Mirkin et al.
13. U.S. Patent Publication 2003/0022470 A1, published Jan. 30, 2003 (“Parallel, Individually Addressable Probes for Nanolithography”) to Liu et al..
14. U.S. Patent Publication 2003/0007242, published Jan. 9, 2003 to Schwartz (“Enhanced Scanning Probe Microscope and Nanolithographic Methods Using Same”).
15. U.S. Patent Publication 2003/0005755 to Schwartz, published Jan. 9, 2003 (“Enhanced Scanning Probe Microscope”).
16. U.S. Regular patent application, Ser. No. 10/366,717 to Eby et al., filed Feb. 14, 2003 (“Methods and Apparatus for Aligning Patterns on a Substrate”).
17. U.S. Regular patent application, Ser. No. 10/375,060 to Cruchon-Dupeyrat et al., filed Feb. 28, 2003 (“Nanolithographic Calibration Methods”).
18. U.S. Patent Publication 2003/049381 A1 to Mirkin et al., published Mar. 13, 2003 (“Methods Utilizing Scanning Probe Microscope Tips and Products Therefor or Produced Thereby”).
19. U.S. Patent Publication 2003/0068446 A1, published Apr. 10, 2003 to Mirkin et al. (“Protein and Peptide Nanoarrays”)
20. U.S. Patent Publication 2003/157254 A1, published Aug. 21, 2003 to Mirkin et al. (“Methods Utilizing Scanning Probe Microscope Tips and Products Therefor or Produced Thereby”).
21. U.S. Patent Publication 2003/162004 A1, published Aug. 28, 2003 to Mirkin, Dravid, Su, Liu (“Patterning of Solid State Features by Direct-Write Nanolithographic Printing”).
22. U.S. Pat. No. 6,635,311 issued Oct. 21, 2003 to Mirkin et al. (“Methods Utilizing Scanning Probe Microscope Tips and Products Therefor or Produced Thereby”).
23. U.S. Pat. No. 6,642,129 issued Nov. 14, 2003 to Liu et al. (“Parallel, Individually Addressable Probes for Nanolithography”).
24. U.S. Pat. No. 6,674,074 issued Jan. 6, 2604 to Schwartz (“Enhanced Scanning Probe Microscope”).
25. U.S. Patent Publication 2004/008330 A1 published Jan. 15, 2004 to Mirkin, Lim (“Electrostatically Driven Lithography”).
26. U.S. Patent Publication 2004/028814 A1, published Feb. 12, 2004 to Mirkin et al. (“Methods Utilizing Scanning Probe Microscope Tips and Products Therefor or Produced Thereby”).
27. U.S. Patent Publication 2004/037959 A1, published Feb. 26, 2004 to Mirkin et al. (“Methods Utilizing Scanning Probe Microscope Tips and Products Therefor or Produced Thereby”).
28. U.S. Pat. No. 6,737,646 issued to Schwartz (“Enhanced Scanning Probe Microscope and Nanolithographic Methods Using Same”).
29. U.S. Patent Publication 2004/119490 A1, published Jun. 24, 2004 to Liu et al. (“Parallel, Individually Addressable Probes for Nanolithography”).
30. U.S. Patent Publication 2004/131843 A1, published Jul. 8, 2004 (“Nanolithography Methods and Products Produced Therefor and Produced Thereby”).
31. U.S. Patent Publication 2004/142106 A1, published Jul. 22, 2004 (“Patterning Magnetic Nanostructures”).
32. U.S. Patent Publication 2004/175631 A1, published Sep. 9, 2004 (“Nanometer-scale engineered structures, methods and apparatus for fabrication thereof, and applications to mask repair, enhancement, and fabrications”).

The invention is further described with use of the following non-limiting working examples.

### WORKING EXAMPLES

### Example 1

### Hot Embossing of an Overt Mark Into a Pharmaceutical Tablet

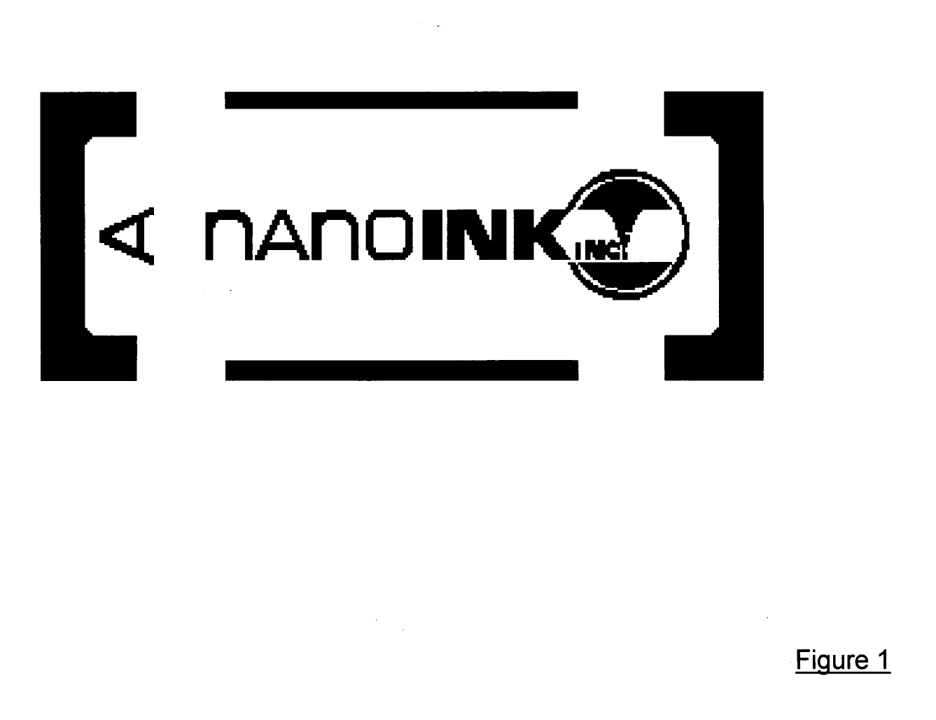

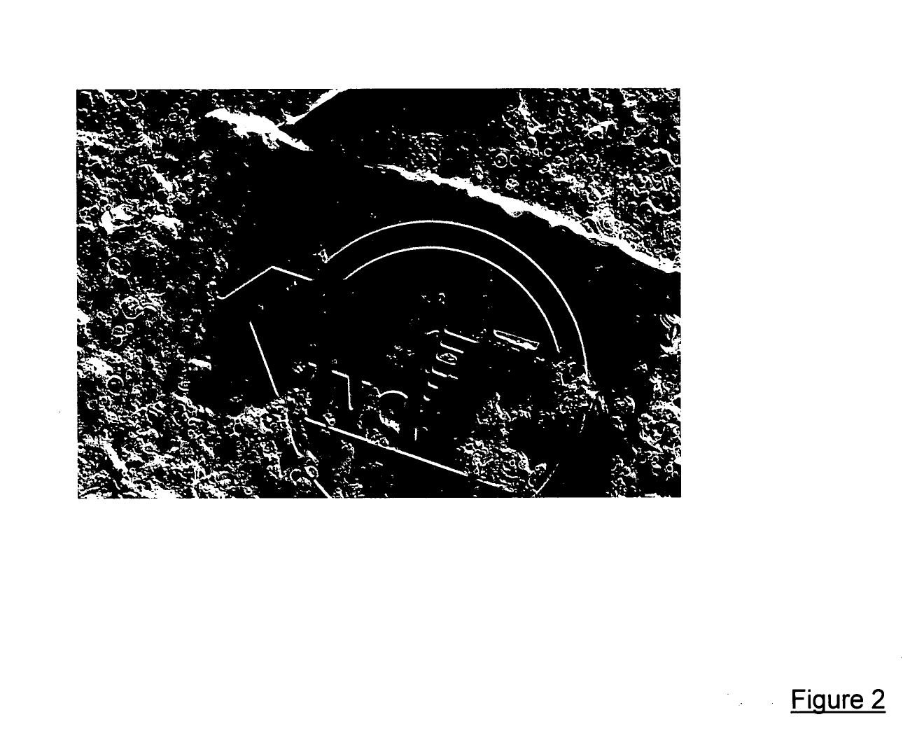

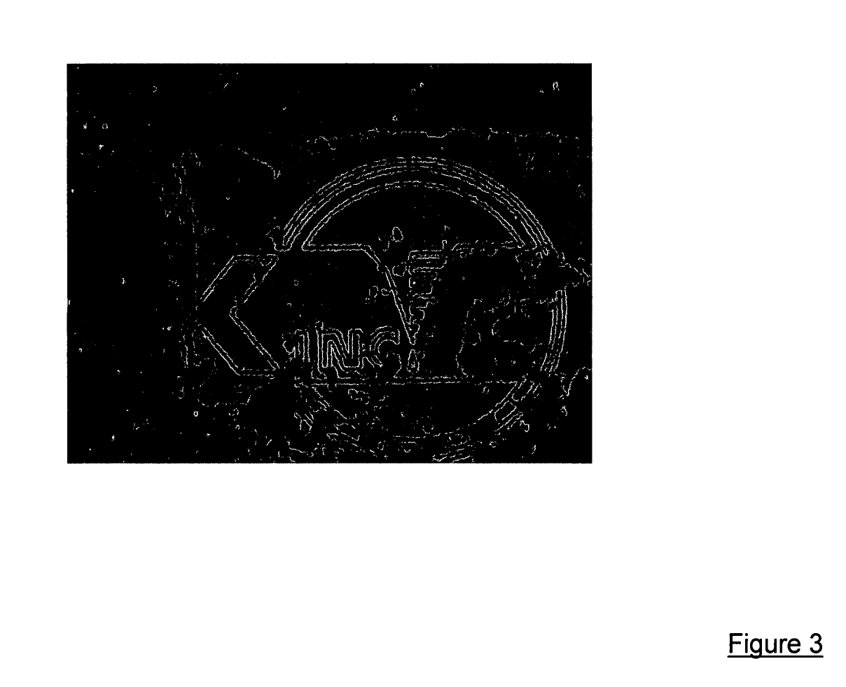

In a first experiment, a silicon stamp shaped as Nanolnk's logo was hot embossed into a pharmaceutical tablet (130° C., 0.5 kg/mm²) for 1 second. FIG. 1 is a schematic diagram of the stamp used for embossing. Darkened areas represent raised features while white areas represent deeper features. Pressure can be and was adapted as desired to, for example, 1.5 kg/mm² or 2 kg/mm². The stamp was fabricated by photolithography, followed by dry etching of the silicon wafer and dicing. The stamp feature depths were 1.8 micron, 2.8 micron, 5 micron and 10 micron for different runs. FIG. 2 shows a scanning electron micrograph of the tablet's surface after embossing, illustrating the fair replication of the stamp features (including the smallest ones). FIG. 3 shows the same feature in an optical microscopy image with concentric illumination.

### Example 2

### Hot Embossing of a Pharmaceutical Tablet with a Commercial Microstructured Surface

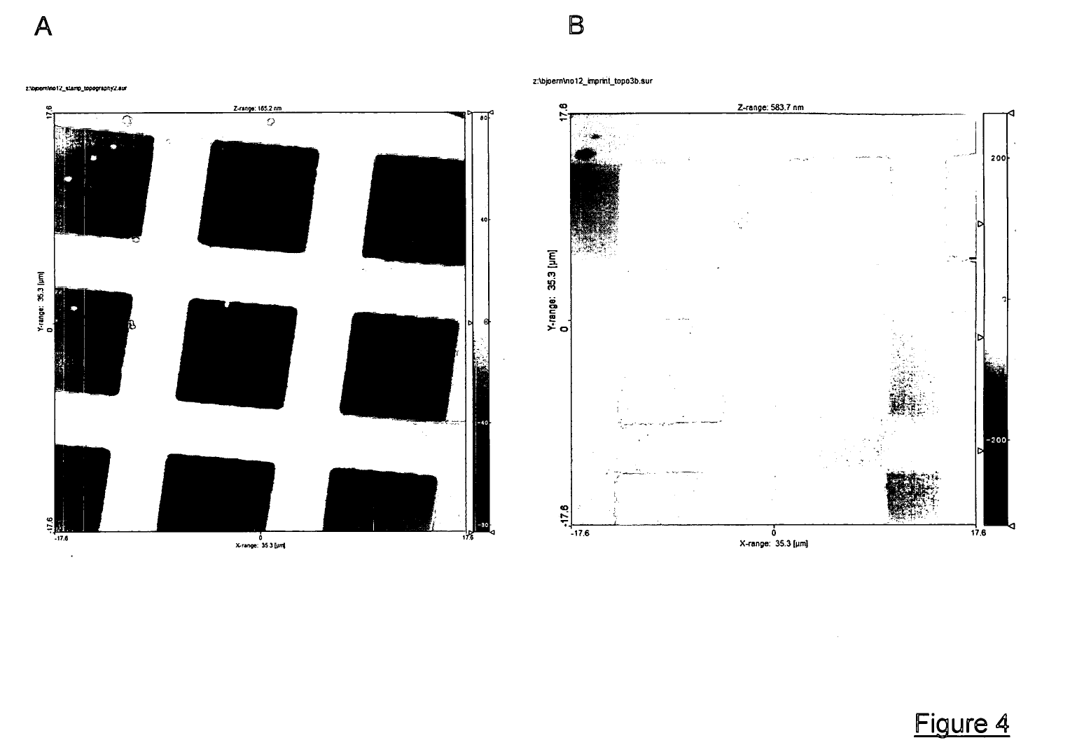

In a second experiment, a commercial microstructured surface, in this example a commercial silicon calibration grating prepared by standard microfabrication methods, was mounted on a custom semi-automated hot embossing machine capable of simultaneously heating said grating and pressing it against a commercial tablet for a controllable duration. For purposes of this application, the inventive features do not reside in the method of making the stamp or the instrument used for stamping. The grating features an array of 10 micron-by-10 micron square recesses with 5 micron spacing between squares in both directions. The recesses are about 150 nm deep. The grating material is single-crystal silicon or single-crystal silicon with a layer of silicon dioxide. The microstructured surface was pressed against a commercial pharmaceutical tablet and the pattern was transferred. FIG. 4-A shows an AFM image of a small part of the microstructured surface. FIG. 4-B shows the resulting transferred pattern on the commercial tablet. The recessed squares on the microstructured surface were transformed to raised squares on the commercial tablet. Analysis of the AFM data showed that the pattern height of 150 nm was well transferred.

In a second step, the tablet was illuminated with a red laser at a grazing angle and its reflection was observed on a white screen. Illuminating the tablet at a location that was not patterned, the laser beam shows a simple reflection with no diffraction effects (5-A). When pointing the laser at the microstructured surface area of the tablet, a diffraction pattern was clearly observed (5-B) to the second order.

The process was repeated with a commercially available hard gelatin capsule, albeit at a lower temperature (105° C.) and pressure. For the purpose of this demonstration, the capsule was opened, emptied of its contents and a fragment of the capsule shell was mounted on a stainless steel disk before imprinting. The embossing can be carried out before capsule filling, for example, at the time of the fabrication of said capsule shell or just afterwards. Embossing can be carried out in a module of the capsule filling machine at the pharmaceutical manufacturing plant between the capsule orienting and capsule filling steps.

The imprinted region was opalescent to the eye when placed on a dark background and under proper natural light illumination in a manner reminiscent of the original grating.

Priority provisional application serial no. 60/637,064 filed Dec. 20, 2004 to Cruchon-Dupeyrat et al. is incorporated by reference in its entirety and provided the following 25 embodiments:

### EMBODIMENT 1

A pharmaceutical composition comprising:

a pharmaceutical composition comprising at least one identification region, said region comprising at least two features, each feature having at least one lateral dimension of about 100 microns or less, the features collectively creating at least one distinctive optical effect that may be readily observed with the naked eye or with the help of a simple device or kit.

### EMBODIMENT 2

The pharmaceutical composition according to embodiment 1, said features having at least one lateral dimension inferior to about 15 micron.

### EMBODIMENT 3

The pharmaceutical composition according to embodiment 2, said features having at least one lateral dimension inferior or equal to about 1.5 micron.

### EMBODIMENT 4

The pharmaceutical composition according to embodiment 3, said features having at least one lateral dimension inferior to about 0.6 micron.

### EMBODIMENT 5

The pharmaceutical composition according to embodiment 1, where the distinctive optical effect is an invariable optical effect.

### EMBODIMENT 6

The pharmaceutical composition according to embodiment 1, wherein said distinctive optical effect comprises a distinctive hue that does not solely result from the presence of a dye or pigment.

### EMBODIMENT 7

The pharmaceutical composition according to embodiment 1, wherein said distinctive optical effect comprises absorbing light, in a way that does not solely result from the presence of a dye or pigment.

### EMBODIMENT 8

The pharmaceutical composition according to embodiment 1, wherein said distinctive optical effect is a variable optical effect.

### EMBODIMENT 9

The pharmaceutical composition according to embodiment 5, wherein at least one identification region forms a hologram.

### EMBODIMENT 10

The pharmaceutical composition according to embodiment 1, wherein at least one overt identification region is opalescent.

### EMBODIMENT 11

The pharmaceutical composition according to embodiment 1, wherein at least one overt region presents a moire effect when observed with the proper device.

### EMBODIMENT 12

The pharmaceutical composition according to embodiment 1, wherein said distinctive optical effect comprises polarizing reflected light.

### EMBODIMENT 13

A pharmaceutical composition according to embodiment 1 further comprising at least one covert identification region, the region having at least one identification feature, the feature having a lateral dimension of about 5 microns or less.

### EMBODIMENT 14

A counterfeiting-proof overt mark within or at the surface of a pharmaceutical composition, said mark comprising at least one engineered collection of micrometer-scale to nanometer-scale patterns, the lateral size of said patterns being inferior to about 100 microns.

### EMBODIMENT 15

The mark according to embodiment 14, wherein said engineered collection is a periodic array.

### EMBODIMENT 16

The mark according to embodiment 15, where the pitch between patterns is inferior to about 1.5 micron.

### EMBODIMENT 17

The mark according to embodiment 15, wherein the periodic array comprises a grating of substantially parallel lines.

### EMBODIMENT 18

The mark according to embodiment 15, wherein said engineered array comprises at least one array of dots or squares.

### EMBODIMENT 19

The mark according to embodiment 14, wherein said engineered collection comprises a pseudo-random distribution of micro- or nanometer-scale patterns.

### EMBODIMENT 20

An object comprising at least one overt identification region, said region comprising at least two features, each feature having at least one lateral dimension inferior to about 30 microns, the features collectively creating a distinctive optical effect that may be observed with the naked eye or with the help of a simple device or kit, said object or composition is selected among the group consisting of medical devices, implants, catheters, stents, syringes, injectable drug vials, pharmaceutical tablet container, excluding bottle seals and labels.

### EMBODIMENT 21

A method for the direct imprinting of a pharmaceutical composition with at least one counterfeiting-proof overt mark without use of ink, the method comprising the steps of:
providing a stamp, which has a surface adapted to form at least one region comprising at least one engineered collection of patterns, the lateral size of said patterns being inferior to about 100 microns;
providing a pharmaceutical composition having a surface;
and contacting the stamp and the pharmaceutical composition.

### EMBODIMENT 22

A method according to embodiment 21 wherein a hard, heated stamp is used.

### EMBODIMENT 23

The method according to embodiment 21, where said surface is contoured, three-dimensional in nature.

### EMBODIMENT 24

A method for printing a pharmaceutical composition having at least one counterfeiting-proof overt mark with use of ink, the method comprising the steps of: providing a stamp coated with ink, which has a surface adapted to transfer ink to form at least one region comprising at least one engineered collection of patterns, the lateral size of said patterns being inferior to about 15 microns;

providing a pharmaceutical composition having a surface;

and contacting the stamp and the pharmaceutical composition, so that the printed region provides at least one distinctive optical effect that may be readily observed with the naked eye or with the help of a simple device or kit.

### EMBODIMENT 25

A pharmaceutical composition comprising:

a pharmaceutical composition having a surface, wherein the surface comprises at least one overt identification region, the overt identification region having at least two identification features, wherein the features each have lateral dimensions of about 100 microns or less and form an overt identification region.

1. A pharmaceutical composition comprising:
a pharmaceutical composition comprising at least one identification region, said region comprising at least two features directly disposed on the pharmaceutical composition without use of an ink or chemical compound and without use of a coating layer adapted to receive the features, each feature having at least one lateral dimension of about 100 microns or less, the features collectively creating at least one optical effect that may be readily observed with the naked eye.

2. The pharmaceutical composition according to claim 1, said features having at least one lateral dimension inferior to about 15 micron.

3. The pharmaceutical composition according to claim 1, said features having at least one lateral dimension inferior or equal to about 1.5 micron.

4. The pharmaceutical composition according to claim 1, said features having at least one lateral dimension inferior to about 0.6 micron.

5. The pharmaceutical composition according to claim 1, where the optical effect is an invariable optical effect.

6. The pharmaceutical composition according to claim 1, wherein the optical effect comprises a distinctive hue that does not solely result from the presence of a dye or pigment.

7. The pharmaceutical composition according to claim 1, wherein the optical effect comprises absorbing light, in a way that does not solely result from the presence of a dye or pigment.

8. The pharmaceutical composition according to claim 1, wherein said the optical effect is a variable optical effect.

9. The pharmaceutical composition according to claim 1, wherein at least one identification region forms a hologram.

10. The pharmaceutical composition according to claim 1, wherein the features collectively are opalescent.

11. The pharmaceutical composition according to claim 1, wherein the features collectively present a moiré effect when observed with the proper device.

12. The pharmaceutical composition according to claim 1, wherein the distinctive optical effect comprises polarizing reflected light.

13. A pharmaceutical composition according to claim 1 further comprising at least one covert identification region, the region having at least one identification feature, the feature having a lateral dimension of about 5 microns or less.

14. A pharmaceutical composition comprising:
a pharmaceutical composition comprising at least one identification region, said region comprising at least two features directly imprinted on the pharmaceutical composition without use of an ink or chemical compound and without use of a coating layer adapted to receive the features, each feature having at least one lateral dimension of about 100 microns or less, the features collectively creating at least one optical effect that may be readily observed with the naked eye or with the help of a device or kit.

15. The pharmaceutical composition according to claim 14, said features having at least one lateral dimension inferior to about 15 micron.

16. The pharmaceutical composition according to claim 14, said features having at least one lateral dimension inferior or equal to about 1.5 micron.

17. The pharmaceutical composition according to claim 14, said features having at least one lateral dimension inferior to about 0.6 micron.
